(19) United States
(12) Patent Application Publication
(10) Pub. No.: US 2017/0052177 A1
(43) Pub. Date: Feb. 23, 2017

(54) COMPETITION ASSAY

(71) Applicant: PharmaDiagnostics NV, Leuven (BE)

(72) Inventors: Wilhelmus Jacobus Laan, Mortsel (BE); Bieke Van De Broek, Leuven (BE); Vanessa Adrienne Therese Bonnard, Uccle (BE)

(21) Appl. No.: 15/306,195

(22) PCT Filed: Apr. 24, 2015
(86) PCT No.: PCT/EP2015/058871

§ 371 Date: Oct. 24, 2016

(30) Foreign Application Priority Data
Apr. 25, 2014 (GB) 1407303.5

(51) Int. Cl. G01N 33/543; G01N 21/552

(52) U.S. Cl. CPC G01N 33/54346; G01N 21/554

(57) ABSTRACT

The present invention relates to a method of determining an interaction between a target compound and a test compound, based on the monitoring of the localized surface plasmon resonance (LSPR) properties of metallic nanoparticles.

### FIELD OF THE INVENTION

The present invention relates to methods and tools for determining an interaction between a target compound and a test compound, based on the monitoring of the localized surface plasmon resonance (LSPR) properties of metallic nanoparticles.

### BACKGROUND OF THE INVENTION

Competition assays, also known as competitive binding assays, are an important tool for determining the concentration of an analyte and/or identifying interactions between two compounds such as a protein and an antibody. In a typical competition assay, a substance competes for labeled versus unlabeled ligand, although there is an increasing need for label-free assays.

A number of label-free assays are based on the use of surface plasmon resonance (SPR), for example as described in US2012/0157328. These assays typically involve the detection of changes in the refractive index at the surface of an SPR detection system as a result of the immobilization of the compounds on a solid surface. Similarly, Warsinke et al. (Analytica Chimica Acta 2005, 550, 69-76) have described methods for the quantification of human tissue inhibitor of metalloproteinases-2 (TIMP-2) using SPR and functionalized gold nanoparticles for signal enhancement. The methods involve the immobilization of TIMP-2 or a protein with high affinity to TIMP-2 to a sensor surface. The presence of TIMP-2 in a test solution influences the interaction between the sensor surface and the functionalized nanoparticles, which is detected via an SPR signal.

WO 2004//042403 relates to methods for the detection of an analyte in a sample involving the use of functionalized nanoparticles which are immobilized on a surface. The methods involve measuring scattered light emitted by individual nanoparticle structures. However, these SPR methods are typically too slow for high-throughput screening. Moreover, the methods described by Warsinke et al. allow for the quantification of a test compound, but do not allow for the screening of a plurality of different test compounds. A further issue with these assays is often the fact that solvents such as dimethylsulfoxide (DMSO) disturb the read-out, which significantly limits the use of such assays as a screening tool. Accordingly, there is a need for improved assays which mitigate at least one of these problems.

### SUMMARY OF THE INVENTION

The present inventors surprisingly found that an LSPR-based competition assay using a nanoparticle suspension can provide a highly reliable tool for high-throughput screening of interactions between a target compound and a test compound. Although LSPR sensing using metal particles is known to be sensitive to the presence of DMSO, the present assays are surprisingly reliable when the test compounds are dissolved in DMSO.

Accordingly, provided herein is are methods of determining an interaction between a target compound and a test compound on nanoparticles which involve contacting the target compound with the test compound in the presence of DMSO. More particularly the methods involve determining an interaction between a target compound and a test compound using nanoparticles whereby the test compound and/or the target compound is provided in a solution comprising at least 50% DMSO.

In particular embodiments, methods of determining an interaction between a target compound and a test compound are provided, comprising the steps of:
(a) providing a suspension of a target definition compound (TDC) conjugated to metal nanoparticles (NPs) (TDC-NP conjugate), wherein said TDC can bind to said target compound;
(b) contacting said suspension comprising said TDC-NP conjugate with said target compound and said test compound, wherein said target compound and/or said test compound are provided in a solution comprising at least 50 w % DMSO; thereby obtaining a liquid mixture comprising between 0.5 w % and 50 w % dimethylsulfoxide (DMSO); and
(c) determining whether said test compound modulates binding of said target compound to said TDC, based on the presence or absence of a change in Localized Surface Plasmon Resonance (LSPR) properties of said TDC-NP conjugate when contacting said suspension comprising said TDC-NP conjugate with said target compound and said test compound.

In certain embodiments, the metal nanoparticles are gold nanorods (GNRs).

In particular embodiments, the target compound is a protein.

In certain embodiments, step (b) of the present method comprises (b1) incubating a solution of said target compound with said test compound; thereby obtaining a pre-incubated target compound solution comprising at least 0.5 w % DMSO; and (b2) contacting said TDC-NP conjugate with said pre-incubated target compound solution.

In particular embodiments, step (c) of the present method comprises (c1) monitoring step (b) by illuminating said nanoparticles with at least one excitation light source and monitoring one or more optical properties of said nanoparticles; and (c2) detecting a change of one or more optical properties of said nanoparticles wherein said change is a result of the presence of an interaction between said target compound and said TDC. In further embodiments, steps (c1) and (c2) are repeated at least once.

In certain embodiments, step (c) comprises correcting the change in LSPR properties of the TDC-NP conjugate for the presence of DMSO in said liquid mixture comprising between 0.5 w % and 50 w % dimethylsulfoxide (DMSO).

In particular embodiments, step (a) comprises (a1) providing a suspension of metal nanoparticles (NPs); (a2) coupling said TDC to a linker molecule; and (a3) conjugation of said TDC to said nanoparticles via said linker molecule, thereby obtaining a suspension comprising said TDC-NP conjugate.

In certain embodiments, the present method further comprises determining the target compound concentration to be used in step (b) via a concentration titration of said TDC-NPs with said target compound.

In particular embodiments, the liquid mixture obtained in step (b) comprises between 0.5 w % and 10 w % DMSO.

In particular embodiments, the solution comprising the test compound further comprises a detergent, preferably a nonionic, cationic and/or zwitterionic detergent, more preferably a nonionic detergent. In further embodiments, the concentration of the detergent in said solution is above the critical micelle concentration.

The methods envisaged herein above can be used, inter alia, to identify a compound which can modulate the interaction between two interacting compounds, such as polypeptides. Accordingly Further provided herein is a method of identifying a compound capable of modulating the interaction between a first polypeptide P1 and a second polypeptide P2, comprising:
(A) providing a suspension of P1 conjugated to metal nanoparticles (NPs) (P1-NP conjugate);
(B) contacting said suspension comprising said P1-NP conjugate with P2 and a test compound; and
(C) determining whether said test compound modulates the interaction between P1 and P2, based on the presence or absence of a change in LSPR properties of said P1-NP conjugate when contacting said suspension comprising said P1-NP conjugate with P2 and said test compound.

In particular embodiments, step (A) comprises (A1) providing a suspension of metal nanoparticles (NPs), wherein said suspension has a pH between (pl-1) and pl, wherein pl is the isoelectric point of P1; (A2) coupling P1 to a linker molecule or coupling a linker molecule to said NPs; and (A3) conjugation of P1 to said nanoparticles via said linker molecule, thereby obtaining a suspension comprising said P1-NP conjugate.

In certain embodiments, the linker molecule is coupled to P1 via a maleimide functional group.

Further provided herein is a kit, more particularly a kit for carrying out the methods envisaged herein. In particular kits are provided comprising a solution comprising a target compound, and preferably comprising at least 50 w % DMSO; and a suspension of a target definition compound (TDC) conjugated to metal nanoparticles (NPs) (TDC-NP conjugate), wherein the TDC can bind to the target compound.

Further provided herein is a computer program on a computer-readable storage medium configured for, when running on a computer, carrying out a method of determining an interaction between a first and a second molecule as envisaged herein. More particularly the computer-readable storage medium is configured for carrying out, when running on a computer, a method comprising the steps of:
loading optical properties obtained by monitoring one or more optical properties of nanorods comprising one or more metals and conjugated with said first molecule and further being incubated with said second molecule;
detecting a change of one or more optical properties of said nanorods; and
determining said interaction by relating said change to the presence of an interaction between said first molecule and said second molecule.

The methods and tools described herein are particularly suitable for high-throughput screening of compounds interacting with biomolecules such as proteins. More particularly, the present methods can allow for compound library screening for determining the binding specificity, kinetics and affinity of a plurality of pre-determined test compounds on a target compound such as a protein of interest. The present inventors further found that such competition assays are surprisingly effective for screening compounds which can modulate the interaction between peptides and/or proteins.

The above and other characteristics, features and advantages of the concepts described herein will become apparent from the following detailed description, which illustrates, by way of example, the principles of the invention.

### BRIEF DESCRIPTION OF THE DRAWINGS

The invention will now be described, inter alia with reference to the accompanying Figures, which are provided by way of example only and should not be considered to limit the scope of the present invention.

FIG. 1 Titration curve for the titration of biotin-GNR with neutravidin, showing ΔRU in function of the concentration of added neutravidin.

FIG. 2 Plot of ΔRU against the amount of biotin and HABA preincubated with a fixed concentration of neutravidin and then added to a biotin-GNR suspension.

FIG. 3 Titration curve for the titration of p53-GNR with MDM2, showing ΔRU in function of the concentration of added MDM2.

FIG. 4 Plot of ΔRU against the amount of p53 (A) and nutlin-3 (B) preincubated with MDM2 and then added to a p53-GNR suspension.

FIG. 5 Plot of the wavelength of maximal absorbance (λmax) of TDC-conjugated nanorods against the amount of various test compounds (1-4) in the absence of detergent.

FIG. 6 Plot of the wavelength of maximal absorbance (λmax) of TDC-conjugated nanorods against the amount of various test compounds (1-4) in the presence of detergent (0.1 v % Triton X-100).

### DETAILED DESCRIPTION OF THE INVENTION

The present invention will be described with respect to particular embodiments but the invention is not limited thereto but only by the claims. Any reference signs in the claims shall not be construed as limiting the scope thereof.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” when referring to recited components, elements or method steps also include embodiments which “consist of” said recited components, elements or method steps.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

As used herein, the term “localized surface plasmon resonance” or “LSPR” relates to methods which detect changes at or near the surface of metal nanoparticles. Typically, these changes are detected by detecting changes in one or more optical properties of the particles. When the metal surfaces of the nanoparticles are excited by electromagnetic radiation, they exhibit collective oscillations of their conduction electrons, known as localized surface plasmons (LSPs). When excited in this fashion, the nanoparticles act as nanoscale antennas, concentrating the electromagnetic field into very small volumes adjacent to the particles. Exceptionally large enhancements in electromagnetic intensity can be obtained this way. The nanoparticles used in the LSPR enable the occurrence of the resonance oscillations.

As used herein, the term “absorbance” refers to the extent to which a sample absorbs light or electromagnetic radiation in the UV, visual or near infrared range of the spectrum. In LSPR changes in refractive index may be detected through monitoring changes in the absorbance. Upon illumination of a sample, changes in the LSPR extinction band of the nanoparticle cause changes in the intensity and/or the wavelength of maximum absorbance.

The term “colloid” refers to a fluid composition of particles suspended in a liquid medium. In representative colloids, the particles therein are between one nanometer and one micrometer in size.

The term “azido” refers to —N₃. The term “amino” by itself or as part of another substituent, refers to —NH₂.

The term “aqueous” as used herein means that more than 50 percent by volume of the solvent is water. Aqueous compositions or dispersions may further comprise organic liquids which are miscible with water.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the features of the claimed embodiments can be used in any combination.

The present invention relates to methods and tools for determining an interaction between a first compound and a second compound, which are herein also referred to as “target compound” and “test compound”, respectively. In particular embodiments, the interaction measured between the test compound and target compound is referred to as “binding”. The term “binding” refers to two molecules associating with each other in a non-covalent or covalent relationship.

The methods described herein involve the use of nanoparticles (NPs), and comprise the steps of (a) Providing a suspension comprising a target definition compound (TDC) conjugated to metal nanoparticles (NPs), wherein the TDC can bind to the target compound. The TDC conjugated to the metal nanoparticles is also referred to herein as “TDC-NP conjugate”. (b) Contacting the suspension comprising the TDC-NP conjugate with the target compound and the test compound; and (c) Determining whether the test compound inhibits binding of the target compound to said TDC, based on the presence or absence of a change in Localized Surface Plasmon Resonance (LSPR) properties of the TDC-NP conjugate when contacting the TDC-NP conjugate with the target compound and the test compound.

In particular embodiments the tools provided herein are specifically adapted to carry out one or more steps of the methods described herein.

This will be explained further herein below.

The methods envisaged herein typically comprise, in a first step (a) providing a suspension of nanoparticles (NPs) to which a TDC is adsorbed, attached, coupled, linked, or bound, generally referred to herein as “conjugated”. The TDC conjugated to nanoparticles is also referred herein as a “TDC-NP conjugate”.

Accordingly, the methods and tools provided herein make use of nanoparticles to which a TDC is conjugated. The nanoparticles can be of any suitable shape and composition and can include but are not limited to nanorods, nanospheres, nanopyramids, nanowires, nanoprisms, nanocubes, nanotetrapods, etc. In particular embodiments, the nanoparticles are nanorods (NRs). NRs can increase the sensitivity of the methods described herein. In further embodiments, the nanorods have an aspect ratio (i.e. length divided by width) ranging between 1.5 and 10, more particularly between 2 and 5. In certain embodiments, the nanorods have a width or diameter between 2 and 20 nm, more particularly between 5 and 18 nm, for example about 15 nm. In particular embodiments, the nanorods have a length between 4 and 60 nm, more particularly between 40 and 50 nm, for example about 48 nm.

The NPs comprise or are made of one or more metals. In certain embodiments, the NPs used in the context of the present invention comprise one or more metals selected from Au, Ag, Cu, Ta, Pt, Pd, and Rh. In certain embodiments, said metal is selected from gold, silver and copper; preferably gold. Particularly good results are obtained if the NPs used are gold nanorods (GNRs).

The nanoparticles provided in the methods and tools envisaged herein are typically provided as a colloid, thus as particles suspended in a solvent. Accordingly, the nanoparticles are not immobilized on a solid substrate, which is particularly useful for high-throughput screening. The solvents suitable for suspending the nanoparticles may depend on the nature of the nanoparticle surface. In preferred embodiments, the nanoparticles are provided with a hydrophilic coating, wherein the solvent may comprise one or more solvents selected from water, ethanol, butanol, isopropanol, acetone, etc. In certain embodiments, the particles are suspended in an aqueous medium.

In particular embodiments, the colloid comprises the nanoparticles in such a concentration that in step (c) of the present method, the colloid has an absorbance at λₘₐₓ between 0.3 and 4, preferably between 0.7 and 1.5. Herein, λₘₐₓ is the wavelength of maximal absorbance of the nanoparticles between 350 and 1000 nm. In certain embodiments, the absorbance of the colloid is between 2 and 27, more particularly between 4.8 and 10.2 at λₘₐₓ, for a path length of 1 cm. Colloids having such absorbance are particularly useful for use with well plates, or other recipients which only require low volumes of the colloid.

The NPs are at least partially coated with a target definition compound (TDC). The TDC is a compound which is known to (specifically) bind to the target compound. The TDC is bound to the NP surface in such a way that it still is able to bind to the target compound. The nature of the TDC is not critical to the methods and tools described herein, and may include small organic molecules, nucleic acids, peptides, proteins, polysaccharides, lipids, or other molecules. In particular embodiments, the TDC may be selected from enzyme inhibitors, protein cofactors, drugs, small molecule antigens of antibodies, and targets of aptamers, proteins, peptides, antibodies. In particular embodiments, the target protein and the TDC are members of a binding couple such as antigen-antibody, receptor-ligand, enzyme-ligand, sugar-lectin, receptor-receptor binding agent, and others.

In particular embodiments, the methods described herein may include the step of conjugating the TDC to the NPs. Methods suitable for conjugating a TDC to NPs are known in the art, and typically involve incubating the nanoparticles in a solution comprising the TDC under conditions which allow the attachment of the TDC onto the nanoparticle surface.

In particular embodiments, the TDC may comprise a metal binding functionality which allows for direct coupling of the TDC to the surface of the metal NPs. A preferred metal binding functionality is a sulfhydryl. Sulfhydryl moieties strongly bind to metal surfaces, particularly to gold surfaces.

In certain embodiments, the TDC may also be coupled indirectly to the NP surface. Indeed, the NPs may be coated with ligand molecules, also referred to herein as “ligands” carrying specific functional groups, such that the surface of the coated NPs exposes these functional groups. The TDC may then be coupled to the NPs via the (functional groups of the) ligands. The functional groups of the ligands may facilitate the conjugation of the TDC to the NP surface, but may also improve other characteristics of the nanomaterial such as solubility and/or stability. For example, if the ligands comprise sulfate, hydroxyl or polyethyleneglycol (PEG) moieties, the stability of the nanoparticle colloids in aqueous media may be improved.

If the conjugation of the TDC to the nanoparticles is performed via functional groups provided on the nanoparticle surface, the functional groups may be activated prior to reaction with the TDC or linker (see further). If the functional group is a carboxyl, the carboxyl may be activated using one or more carboxyl activating groups. Suitable carboxyl activating groups include, but are not limited to, carbodiimide reagents, phosphonium reagents, uranium, and carbonium reagents, as is known by the skilled person.

In particular embodiments, the TDC may not comprise a metal binding functionality, or a functional group which can react with the functional groups exposed on the NP surface. In such cases, the TDC may be coupled to the NP surface or the ligands indirectly, more particularly via a linker molecule, which is also referred to herein as “linker”. Linker molecules may also be used in other cases wherein direct coupling of the TDC to the NP surface or to the ligands is not possible or desired, e.g. to improve the access of the target molecule to the TDC when conjugated to the particles.

The order in which the linker molecule is coupled to the NPs and the TDC is not critical. Thus, the linker may first be coupled to the NPs or to the ligands provided on the NPs followed by coupling of the TDC to the linker, or vice versa. In preferred embodiments, the TDC may first be coupled to the linker molecule, followed by coupling the linker molecule to the NP. More particularly, in specific embodiments step (a) of the present methods may comprise:
(a1) providing a suspension of metal nanoparticles (NPs);
(a2) coupling the TDC to a linker molecule; and
(a3) conjugating the TDC to the nanoparticles via the linker molecule, thereby obtaining a suspension comprising a TDC-NP conjugate.

A variety of linker molecules is known to those of skill in the art and typically includes bi-functional molecules. Generally, such linker molecules comprise a spacer group terminated at one end with a first portion capable of coupling to the nanoparticles (e.g. via a metal binding functionality, or via binding to the ligands provided on the nanoparticle surface) and at the other end a second portion which is a functional group capable of forming a covalent bond to the TDC.

Spacer groups of interest possibly include aliphatic and unsaturated hydrocarbon chains, spacers containing hetero-atoms such as oxygen (ethers such as polyethylene glycol) or nitrogen (polyamines), peptides, carbohydrates, cyclic or acyclic systems that may possibly contain hetero-atoms. Generally, short spacer groups are preferred as they typically result in a stronger LSPR signal. On the other hand, a spacer group which is too short may be insufficient to stabilize the nanoparticles in suspension, in particular when the linker directly binds to the NP surface via a metal binding functionality. Preferred spacer groups comprise a hydrocarbon chain with 6 to 18 and preferably 6 to 12 carbon atoms; or a polyethyleneglycol (PEG) chain of 2 to 5 ethylene glycol monomers, preferably 2 or 3 monomers.

Potential functional groups capable of covalently binding the TDC include nucleophilic functional groups (amines, hydroxyls, sulfhydryls, azides, hydrazides), electrophilic functional groups (alkynyles, carboxyls, aldehydes, esters, vinyl ketones, epoxides, isocyanates, maleimides), functional groups capable of cycloaddition reactions, forming disulfide bonds, or binding to metals. Specific examples include primary and secondary amines, maleimides, hydroxamic acids, N-hydroxysuccinimidyl esters, N-hydroxysuccinimidyl carbonates, oxycarbonylimidazoles, nitrophenylesters, trifluoroethyl esters, glycidyl ethers, and vinylsulfones.

In particular embodiments, the linker is provided with a maleimide functional group. Maleimide functional groups are particularly suitable for conjugating a TDC to the nanoparticles, wherein the TDC is a protein or peptide containing a sulfhydryl (from cysteine) which is available for binding. Many proteins and peptides only contain a single available sulfhydryl group. Accordingly, the coupling of the TDC to the nanoparticles via a linker having a maleimide functional group may allow for a uniform and oriented coupling of the protein or peptide to the nanoparticles, which can improve the reliability of the present methods. In specific embodiments, the linker may be provided with a maleimide functional group (for coupling to the TDC) and an amine group (for binding to carboxylic acid functional groups provided on the nanoparticle surface).

In certain embodiments, the functional group for coupling the linker to the nanoparticles may be a metal binding functionality. A preferred metal binding functionality is sulfhydryl. In preferred embodiments, the functional group for coupling the linker to the nanoparticles is a functional group which can covalently bind to a functional group provided on the (ligands of the) nanoparticles. Indeed, as described above, the surface of the metal nanoparticles provided in (a1) may be provided with one or more functional groups. Preferably, the one or more functional groups are selected from amino, azido, alkynyl, carboxyl, hydroxyl and carbonyl.

In specific embodiments, the nanoparticle surface is provided with carboxyl groups. Carboxyl groups are especially useful for binding proteins, because an activated carboxyl group can react with an amine moiety of a protein, thereby forming an amide bond. In further embodiments, the nanoparticles are at least partially coated with a mercaptocarboxylic acid. The sulfhydryl moiety of the mercaptocarboxylic acid can bind to a metal atom of the nanoparticle surface via chemisorption, while the carboxyl moiety can be used to bind to molecules such as proteins.

In certain embodiments, the functional groups provided on the first and/or second portion of the linker may allow a coupling mechanism as used in Click Chemistry. For example, the functional groups may comprise an azide or an alkyne, thereby allowing an azide alkyne Huisgen cycloaddition using a Cu catalyst at room temperature, as known by the person skilled in the art. An azide functional group further provides the possibility of Staudinger ligation, which typically involves reaction between an azide moiety with a phosphine or phosphate moiety.

In particular embodiments, the TDC-NP conjugate is further contacted with a blocking reagent which reacts with the remaining unreacted functional groups which may be present on the TDC-NP conjugate. This can be done to prevent nonspecific binding of the target compound to the unreacted functional groups. Such blocking reagents are known in the art.

The blocking reagent is typically chosen such that it does not significantly interact with the substances that shall be tested with the conjugated nanomaterial, in particular the target compound. In particular embodiments, the blocking reagent is further chosen such that it contributes to a good solubility of the conjugated nanoparticles in one or more solvents, for example by adding charge, hydrophilicity or steric hindrance.

If the functional group is a carboxyl, then the blocking reagent is a carboxyl blocking reagent, for example a reagent comprising an amino functional group. Suitable carboxyl blocking reagents include but are not limited to Bovine Serum Albumin (BSA), Ovalbumin, and an amino functionalized polyethylene glycol.

If the functional group is an azide, potential blocking reagents are molecules comprising a phosphine or alkyne moiety. If the functional group is an alkyne or phosphine, potential blocking reagents are molecules comprising an azide moiety. Examples of such molecules are modified proteins or peptides which do not significantly interact with the target compound.

In particular embodiments of the methods and tools described herein, the TDC may be conjugated to the NPs in a controlled way, such that the amount of said TDC conjugated to said NPs is below 70% of the amount required for full coverage of said NPs with said TDC, more preferably below 50% of full coverage. This may enhance the LSPR signal upon binding of the TDC to the target compound, in particular at low concentrations of the target compound. The term “full coverage” as used herein refers to the maximal amount of the TDC that can be conjugated or attached to a nanoparticle as a monolayer around said nanoparticle. Full coverage may be obtained by exposing the nanoparticles to a large excess of the TDC in conditions suitable for coating the nanoparticles. The optimal amount of the TDC may be determined via a titration experiment which may involve titration of a fixed amount or concentration of TDC-NPs with a variable amount or concentration of target compound. When plotting optical properties such as λmax or ΔRU (see further) against the amount or concentration of added TDC, the optical properties will change with increasing amount of TDC until full coverage of the nanoparticles is obtained.

The optimal amount may depend on the characteristics of the nanoparticles, such as size and shape, and the TDC and/or target compound. In particular embodiments, the amount of the TDC conjugated to the nanoparticles is below 70%, preferably below 50%, of the amount required for full coverage, wherein the nanoparticles are nanorods with a length between 40 and 60 nm and a diameter between 10 and 20 nm.

If the TDC is conjugated to the nanoparticles via the functional groups of the ligands as described above, the amount of functional groups provided on the nanoparticle surface determines the maximal amount of the TDC that can be conjugated to the nanoparticles. Thus, it can be ensured that less than full coverage of the nanoparticles by the TDC is obtained by limiting the amount of functional groups, for example by coating the NPs with a mixture of ligands of which some do and others do not comprise the required functional group for conjugating the TDC to the NPs.

Additionally or alternatively, less than full coverage may also be obtained by only letting a certain fraction of the functional groups provided on the nanoparticle surface react with the TDC. The required amount of the TDC to reach the desired coverage may be found by a titration experiment. After conjugation of the TDC to the nanoparticles, the non-reacted functional groups present on the nanoparticles may be reacted with (an excess of) a blocking reagent, in order to avoid nonspecific binding and/or a reduced stability of the nanoparticle conjugate. Alternatively, a certain fraction of the functional groups provided on the nanoparticles may first be reacted with a blocking reagent, followed by reacting the non-blocked functional groups with (an excess of) the TDC. Again, a concentration titration may be performed first to determine the optimal amount of blocking reagent required for blocking a specific part of the functional groups provided on the nanoparticles.

The methods described herein typically comprise in a step (b), contacting or incubating the suspension comprising the TDC-NP conjugate with the target compound and the test compound, thereby obtaining a mixture comprising the TDC-NP conjugate, the target compound, and the test compound. Preferably, the target compound and test compound are provided in a (relatively) purified form in a fluid composition, for example a (buffered) solution in water and/or DMSO.

The present methods may be used for high-throughput screening of test compound libraries. Thus, in certain embodiments, the suspension may be contacted with a plurality of test compounds; thereby obtaining a plurality of mixtures each comprising the TDC-NP conjugate, the target compound, and one of the test compounds. In certain embodiments, the compound library is provided as solutions of test compounds in a well-plate.

In practice, test compounds are often dissolved in a solvent which is or comprises dimethylsulfoxide (DMSO). Additionally or alternatively, the target compound may be dissolved in a solvent comprising DMSO. The relatively high refractive index of DMSO compared to solvents as water can be problematic in surface plasmon resonance (SPR) based assays as described in US2012/0157328, as the signal measured therein varies with the refractive index of the medium. The present inventors surprisingly found that the presence of DMSO is not problematic for the NP-based methods described herein. Thus, the test compound and/or target compound need not be transferred to another solvent than DMSO prior to contacting with the TDC-NP conjugate.

Accordingly, in particular embodiments, the test compound and/or the target compound is provided as a solution comprising at least 50 w % (percent by weight) DMSO, wherein the solution may be mixed with the (suspension comprising the) TDC-NP conjugate and the (solution comprising the) target compound as such. In certain embodiments the test compound is provided as a solution comprising at least 50 w % DMSO, preferably at least 75 w % DMSO, more preferably at least 90 w % DMSO.

As one or more of the TDC-NP conjugate, the test compound, and the target compound may not be dissolved or suspended in DMSO, the resulting liquid mixture comprising the TDC-NP conjugate, the test compound, and the target compound may comprise a lower amount of DMSO. Typically, this liquid mixture comprises between 0.5 w % and 50 w % DMSO, between 1 w % and 50 w % DMSO, or even between 5 w % and 50 w % DMSO. DMSO concentrations below 50 w % may be preferred for preserving the stability of the target compound. For example, many proteins are not stable in solvents comprising more than 50 w % DMSO. In certain embodiments, the liquid mixture comprises at most 40 w % DMSO, preferably at most 20 w % DMSO, most preferably at most 10 w % DMSO.

In particular embodiments, the target compound is pre-incubated with the test compound prior to contacting the TDC-NP conjugate with the target compound and test compound. The pre-incubation can significantly shorten the amount of time needed for reaching equilibrium after contacting the TDC-NP conjugate with the test compound and the target compound. Thus, in particular embodiments, the present methods may comprise in a step (b):
(b1) incubating a solution of the target compound with the test compound; thereby obtaining a pre-incubated target compound solution optionally comprising at least 0.5 w % DMSO; and
(b2) contacting the TDC-NP conjugate with the pre-incubated target compound solution.

The incubation time, i.e. the time between steps (b1) and (b2) typically is at least 1 minute, preferably at least 5 minutes, most preferably at least 10 minutes, for example between 15 minutes and 60 minutes.

The optimal amount of target compound to be used in step (b) may depend on various factors, in particular the concentration of NPs in the suspension and the average amount of TDC molecules bound to the NPs. A suitable amount for the target compound may be found via a titration experiment, wherein the LSPR properties of the TDC-NPs are monitored with increasing amounts of added target compound. More particularly, the titration may be monitored via the measurement of the absorbance of the nanoparticles. In particular embodiments, the titration may involve determining ΔRU, i.e. (OD(λ_{max,blank}+80)/OD(λ_{max,sample}))−(OD(λ_{max,blank}+80))/OD(λ_{max,blank})) for each amount of target compound added. Herein, “OD(x)” refers to the optical density at wavelength x (in nm), and λₘₐₓ refers to the wavelength of maximal absorbance of the TDC-NP conjugate. A suitable amount of target compound is an amount which results in a detectable change of the LSPR properties of the conjugate, but which does not saturate the available TDC binding sites (i.e. below the plateau in the plot of ΔRU vs. the amount of added target compound). In certain embodiments, the titration may involve determining Δλₘₐₓ, i.e. the change in the λₘₐₓ for each amount of target compound added. λₘₐₓ refers to the wavelength of maximal absorbance of the TDC-NP conjugate, i.e. the wavelength x for which OD(x) reaches a maximum. A suitable amount of target compound is an amount which results in a detectable change of the LSPR properties of the conjugate, but which does not saturate the available TDC binding sites (i.e. below the plateau in the plot of λₘₐₓ vs. the amount of added target compound).

Preferably the raw absorbance data are processed prior to determining ΔRU and/or Δλₘₐₓ as described above. Data processing may be based on curve fitting (like polynomials or any other representative curve like Gaussian or Lorentzian curves, preferably in a predefined neighborhood, e.g. around a maximum, or even a model, being representative for the resonance phenomena used) and use of the fitted curve instead of the raw data.

The methods envisaged herein typically comprise in a step (c), the determination whether the test compounds modulate (e.g. inhibit) binding of the target compound to the TDC, based on the presence or absence of a change in Localized Surface Plasmon Resonance (LSPR) properties of the TDC-NP conjugate when contacting the (suspension comprising the) TDC-NP conjugate with the target compound and the test compound.

Indeed, when the test compound does not interact with the target compound (or when the test compound binds to the target compound but does not compete with TDC because it does not interact with the binding site of the TDC), the target compound will bind to the TDC which is conjugated to the nanoparticles. The proximity of the target compound to the conjugate changes the refractive index surrounding the nanoparticles, which will lead to a detectable change in the LSPR properties of the TDC-NP conjugate. On the other hand, if there is an interaction between the test compound and the target compound wherein the test compound competes with the TDC for binding to the target compound, the target compound will not bind to the TDC, or only a reduced amount will bind. Accordingly, no change or a minor change in the LSPR properties of the TDC-NP conjugate will be detected.

Preferably, the change in LSPR properties of the TDC-NP conjugate is detected by measuring one or more optical properties of the conjugate in the presence and absence of the target compound. Moreover, the present methods typically involve measuring one or more macroscopic optical properties of the suspension comprising the conjugate. Accordingly, the average optical properties of the nanoparticles is measured, rather than measuring the optical properties of single particles.

More particularly, the present methods may comprise in a step (c):
(c1) monitoring step (b) by illuminating the TDC-NP conjugate with at least one excitation light source and monitoring one or more optical properties of the conjugate; and
(c2) detecting a change of one or more optical properties of the TDC-NP conjugate wherein said change is a result of the presence of an interaction between the target compound and the TDC.

The light source used in (c1) typically emits light or radiation at one or more wavelengths between 350 and 1000 nm. In particular embodiments an excitation light source is used which emits light or radiation comprising between approximately 1 nanowatt and 100 watts of power. In more particular embodiments the excitation light source is a (xenon) flash lamp or a laser.

In particular embodiments step (c1) is repeated at least once and said step (c2) is applied to an averaged optical property obtained from said repetition. In certain embodiments both step (c1) and step (c2) are repeated at least once, wherein the final detection of a change of one or more optical properties is based on an average of the detection obtained from each execution of step (c1), followed by (c2). These embodiments can be combined, in that averaging over a plurality of measurements, to obtain a plurality averaged optical properties, followed by detection over each of said plurality of averaged optical properties, whereby such detections need to be combined to have a final detection. In this combined embodiment the effort used to increase accuracy is spread over improving the raw data itself versus improvement of the detection of change.

The term “detecting” as used herein means to ascertain a signal (or a change therein), either qualitatively or quantitatively. The methods described herein comprise the step of detecting a signal, more particularly a change in signal at one or more wavelengths. The terms “monitoring”, “determining”, “measuring”, “assessing”, “detecting” and “evaluating” are used interchangeably to refer to any form of measurement, and includes not detecting any change. Said measurement may include both quantitative and qualitative determinations either relative or absolute and also include determining the amount of something present, as well as determining whether it is present or absent.

In preferred embodiments, an optical property of the conjugate which is monitored is the absorbance of the conjugate. Indeed, the conjugation of the target compound to the TDC-NP conjugate leads to a difference in refractive index around the nanoparticles and thereby to a redshift of the λₘₐₓ that can be detected by reading an absorbance spectrum.

In particular embodiments, the change in absorbance properties is expressed as ΔRU, as defined above. In particular embodiments, the absorbance of the conjugate is measured at two or more wavelengths between 350 and 1000 nm. Measurement at two or more wavelengths can allow for obtaining more accurate data. In particular embodiments, these wavelengths are discrete wavelengths within that range. Preferably the raw OD(x) data are processed before use in any of the above embodiments. As an example, such processing may be based on curve fitting and use of the fitted curve instead of the raw data.

In particular embodiments, steps (c1) and (c2) may be performed more than once, preferably after regular time intervals. This may allow for determining whether the mixture has reached equilibrium. Indeed, the LSPR signal may change as long as the mixture advances towards its equilibrium, only to become stable when equilibrium has been reached. It is preferred that the mixture reaches equilibrium, as this allows for a more precise quantification of the interaction between the test compound and the target compound. It is noted that the methods described herein do not suffer from bleaching of the TDC-NP conjugate, in contrast with other methods such as fluorescence-based assays. Accordingly, there is practically no limit on the amount of iterations of steps (c1) and (c2) which can be performed.

In particular embodiments, steps (c1) and (c2) are reiterated regularly, with a time interval between successive iterations between 0.5 seconds and 20 minutes. If there are multiple samples, e.g. provided in a multi-well plate, a new iteration preferably starts when the previous iteration has been completed for all samples. In such embodiments, a typical time interval is about 15 minutes. Preferably, the iteration is terminated when the measured LSPR properties are stable or when a predefined time limit has expired, whichever occurs first.

As indicated above, the present methods can be used even when the solvents used comprise DMSO. Preferably, step (c) of the methods described herein may comprise correcting the observed change in LSPR properties of the TDC-NP conjugate for the presence of DMSO. The correction step may involve correcting the measured change in LSPR properties of the TDC-NP conjugate, by subtracting the contribution of DMSO to the change. In preferred embodiments, the correction may involve comparing the optical properties of the sample with the optical properties of a reference (blank) sample comprising the TDC-NP conjugate and target compound in the same solvent (comprising DMSO) but without the test compound.

The methods of the present invention are of particular interest in the context of screening methods. Thus in particular embodiments the present invention provides screening methods wherein detection is performed according to the present invention. In further embodiments, the methods are high-throughput screening methods, more particularly methods which are at least in part carried out in a high-throughput screening device.

More particularly, the subject methods may be used to screen for compounds that modulate the interaction between the target molecule and the TDC. The term modulating includes both decreasing (e.g. inhibiting) and enhancing the interaction between the two molecules.

The methods described herein are particularly suitable for identifying test compounds that can modulate the interaction between a first (poly)peptide (P1) and a second (poly)peptide (P2). The term “polypeptide” as used herein includes proteins. In such embodiments, the effect of the test compound on the interaction between P1 and P2 can be determined using the methods described herein, wherein P1 can be selected as the target compound and P2 as the TDC, or vice versa. Thus, further provided herein are methods of identifying a compound capable of modulating the interaction between two (poly)peptides and/or proteins (P1 and P2), comprising:
(A) providing a suspension of the first (poly)peptide (P1) conjugated to metal nanoparticles (NPs) (P1-NP conjugate);
(B) contacting the suspension comprising the P1-NP conjugate with the second (poly)peptide (P2) and a test compound; and
(C) determining whether the test compound modulates the interaction between P1 and P2, based on the presence or absence of a change in LSPR properties of the P1-NP conjugate when contacting the suspension comprising the P1-NP conjugate with P2 and the test compound.

The details of steps (a), (b), and (c) as described above apply, mutatis mutandis, to steps (A), (B), and (C). In particular, step (B) may include determining the optimal amount of P2 to be added to the conjugate, via a titration experiment as described above.

In particular embodiments, the method may include the selection of a suitable ionic strength for the suspension comprising the P1-NP conjugate. In particular embodiments, this may include selecting a maximal value for the ionic strength. It is preferred that an ionic strength below the maximal value is respected prior to and after contacting with P2 and the test compound. The inventors have found that for a large number of proteins an optimal stability of the P1-NP conjugate can be obtained by using a suspension having an ionic strength below 20 mM. Without wishing to be bound by theory, it is believed that an ionic strength below the maximal value avoids shielding of the charges on the nanoparticles and/or on the polypeptides. On the other hand, some P1-P2 interactions may require a minimal ionic strength. Accordingly, in preferred embodiments, the ionic strength of the suspension may be between 5 mM and 20 mM, for example about 10 mM. The ionic strength of the suspension can be increased through the addition of salts which form ions when dissolved in the solvent of the suspension, as is known by the skilled person.

The present inventors have found that certain test compounds can cause a change in the measured LSPR signal of the TDC-NP conjugate at high test compound concentrations, while not causing a significant change in the LSPR signal at lower test compound concentrations. Without wishing to be bound by theory, the present inventors believe that this is caused by formation of test compound aggregates resulting from a limited solubility of the test compound in the nanoparticle suspension. Such aggregation can lead to unwanted background signals. The present inventors have found that when the solution comprising the test compound comprises a detergent, such background signals can be suppressed. Accordingly, in particular embodiments, the solution comprising the test compound further comprises at least one detergent.

In particular embodiments, the detergent is a nonionic, cationic and/or zwitterionic detergent. It will be understood to the skilled person that reference herein to the use of a nonionic, cationic and/or zwitterionic detergent includes the use of combinations of different nonionic, cationic and/or zwitterionic detergents. In preferred embodiments, the detergent is a nonionic detergent.

The term “nonionic detergent” as used herein refers to a detergent which does not have any ionic groups. In embodiments of the methods of the invention, the nonionic detergent is selected from the group comprising octylphenol ethoxylates, polysorbates, glucamines, lubrol, Brij®, Nonidet®, Pluronic®, Genapol® and Igepal®. In particular embodiments, the polysorbate is chosen from the group comprising polysorbate 20, polysorbate 40, polysorbate 60, polysorbate 65, polysorbate 80 and polysorbate 85.

In preferred embodiments, the nonionic detergent is an octylphenol ethoxylate. In particular embodiments, the octylphenol ethoxylate is selected from the group comprising TRITON® X-15, TRITON® X-35, TRITON® X-45, TRITON® X-100, TRITON® X-102, TRITON® X-114, TRITON X-165 (70%), TRITON® X-305 (70%), TRITON® X-405 (70%) and TRITON® X-705 (70%). In particular embodiments, the glucamine is selected from the group comprising of N-octanoyl-N-methylglucamine (MEGA-8), N-nonanoyl-N-methylglucamine (MEGA-9) and N-decanoyl-N-methylglucamine (MEGA-10).

The term “cationic detergent” as used herein refers to a detergent with a positive ionic charge. In embodiments of the methods of the invention, the cationic detergent is selected from hexadecyltrimethyl ammonium bromide (CTAB) or trimethyl(tetradecyl) ammonium bromide (TTAB).

The term “zwitterionic detergent” as used herein refers to a detergent which has ionic groups, but no net charge. In embodiments of the methods of the invention, the zwitterionic detergent is selected from the group comprising amidosulfobetaines, alkylbetaines and ammonio propanesulfonates. In preferred embodiments, the zwitterionic detergent is selected from the group comprising amidosulfobetaine-14, amidosulfobetaine-16, 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), 3-(4-heptyl)phenyl-3-hydroxypropyl)dimethylammoniopropanesulfonate (C7BzO), EMPIGEN® BB, 3-(N,N-dimethyloctylammonio) propanesulfonate inner salt, 3-(decyldimethylammonio) propanesulfonate inner salt, 3-(dodecyldimethylammonio) propanesulfonate inner salt, 3-(N,N-dimethylmyristylammonio) propanesulfonate inner salt, 3-(N,N-dimethylpalmitylammonio) propanesulfonate inner salt, 3-(N,N-dimethyloctadecylammonio) propanesulfonate inner salt.

In preferred embodiments, the detergent is used in a concentration equal to or above the critical micelle concentration (CMC). The CMC of a detergent is the concentration at which the detergent forms higher aggregates, so-called micelles. The CMC can be determined via titration and by the determining the jump in physical properties such as for example the surface tension, the osmotic pressure, the equivalent conductivity, the interfacial tension and/or the density. Each of these parameters can be measured with known methods. Preferably, the detergent is used in a concentration which is equal to or above the CMC, before and after contacting the solution comprising the test compound and detergent with the solution comprising the target compound and the suspension comprising the TDC-NP. In particular embodiments, the concentration of the detergent in the solution comprising the test compound is between 1 time and 20 times the CMC, more particularly between 5 times and 10 times the CMC. It is not excluded that in other embodiments, the detergent may be used in a concentration below the CMC, for example between 10% and 95% of the CMC.

The inventors have further found that if step (A) includes conjugating P1 to the nanoparticles, the nanoparticle suspension should preferably be buffered at a pH above (pl-1), wherein pl is the isoelectric point of P1, this is the pH at which P1 or its surface carries no net electrical charge. More preferably, the nanoparticle suspension is buffered at a pH between (pl-1) and pl. This is particularly advantageous when the nanoparticles are provided by functional groups which carry negative charges, such as carboxyl groups.

Thus, in certain embodiments, step (A) may comprise:
(A1) providing a suspension of metal nanoparticles (NPs), wherein said suspension has a pH above (pl-1), and preferably between (pl-1) and pl, wherein pl is the isoelectric point of P1;
(A2) coupling P1 to a linker molecule, or coupling a linker molecule to the NPs; and
(A3) conjugation of P1 to said nanoparticles via said linker molecule, thereby obtaining a suspension comprising the P1-NP conjugate.

The details of steps (a1), (a2), and (a3) as described above apply, mutatis mutandis, to steps (A1), (A2), and (A3).

The pH of the nanoparticle suspension in steps (B) and (C) may be the same or different than the pH range used in step (A).

In practice, the purification of peptides and proteins may involve providing the peptides and proteins with a tag such as a Histidine-tag (His-tag), which may bind non-specifically to the metal surface of the nanoparticles. In order to prevent such non-specific binding, a small amount of imidazole may be added to the P1-NP suspension. The imidazole will then compete with the His-tag for the metal. Preferably, imidazole is added to the suspension to a concentration between 5 and 50 mM.

The present application further provides tools for carrying out one or more steps of the methods described herein.

More particular, provided herein is a kit comprising
a target compound;
a suspension of a TDC-NP conjugate as described herein wherein the TDC can bind to the target compound;
optionally, instructions for use of the TDC-NP conjugate in one or more of the methods described herein.

In certain embodiments, the kit may further comprise one or more test compounds as described herein. More particularly, the kit may comprise a plurality of test compounds, which may be provided in a multi-well plate. In certain embodiments, the one or more test compounds each are provided as a solution comprising the test compound in at least 50 w % DMSO.

Further provided herein is a computer program, preferably on a computer-readable storage medium, configured for at least partially carrying out the methods of determining an interaction between a target compound and a test compound as disclosed herein.

In particular embodiments, the computer program may be configured to control an apparatus such as a robot, for contacting the suspension comprising the TDC-NP conjugate with the target compound and a plurality of test compounds, e.g. in a multi-well plate.

In certain embodiments, the computer program may be configured to control an apparatus for measuring the LSPR properties of the TDC-NP conjugate, such as a spectrophotometer.

The computer program may further be configured to process the data obtained via the measurements, and to use the data to determine or quantify the interaction between the test compounds and the target compound. Thus, in particular embodiments, computer programs are provided, which, when running on a computer, determine or quantify the interaction between the test compounds and the target compound.

In specific embodiments, the computer program is configured for determining whether the mixture comprising the TDC-NP conjugate, the target compound, and the test compound has reached equilibrium. This can be done by performing multiple reads, e.g. by repeating steps (c1) and (c2) as described above.

Further provided herein is a computer program configured for carrying out a method of determining an interaction between a first and a second molecule, the method comprising:
loading optical properties obtained by monitoring one or more optical properties of nanorods comprising one or more metals and conjugated with said first molecule and further being incubated with said second molecule;
detecting a change of one or more optical properties of said nanorods; and
determining said interaction by relating said change to the presence of an interaction between said first molecule and said second molecule.

In particular embodiments, the computer program may be configured for carrying out a method of determining an interaction between a target compound and a test compound, said method comprising:
loading Localized Surface Plasmon Resonance (LSPR) properties obtained from a suspension of a target definition compound (TDC) conjugated to metal nanoparticles (NPs) (TDC-NP conjugate), wherein said TDC can bind to said target compound, whereby said suspension comprising said TDC-NP conjugate with said target compound is contacted with said test compound; thereby obtaining a liquid mixture;
determining the presence or absence of a change in Localized Surface Plasmon Resonance (LSPR) properties of said TDC-NP conjugate when contacting said suspension comprising said TDC-NP conjugate with said target compound and said test compound; and
determining whether said test compound modulates binding of said target compound to said TDC, based on said presence or absence of a change in Localized Surface Plasmon Resonance (LSPR) properties.

In certain embodiments, the computer program may be configured for carrying out a method of identifying whether a compound is capable of modulating the interaction between a first polypeptide P1 and a second polypeptide P2, said method comprising:
loading LSPR properties obtained from a P1-NP conjugate when contacting a suspension of P1 conjugated to metal nanoparticles (NPs) (P1-NP conjugate) comprising said P1-NP conjugate with P2 and a test compound;
determining the presence or absence of a change in LSPR properties of said P1-NP conjugate when contacting said suspension comprising said P1-NP conjugate with P2 and said test compound; and
determining whether said test compound modulates the interaction between P1 and P2, based on said presence or absence of a change in LSPR properties.

Further provided herein is a computer program, also referred herein as “guiding program”, which is configured for carrying out a method of step-by-step interactive guiding any of the determining or identifying methods described above, by providing
an indication of the amount of materials needed; and/or
guidance through the setup of the method (experiment); and/or
   input for any of the above computer program methods of loading, determining LSPR properties changes and interpretation thereof. In particular embodiments, the guiding program may be configured to execute a method for assisting at least the preparation of a method of determining an interaction between a first and a second molecule, the assisting method comprising;
loading one or more of the following input parameters such as the properties and amounts of the nanorods, said first molecule and said second molecule; and
computing (and output or display) from those input parameters one or more of the remaining parameters needed to start the determining method.

In certain embodiments, the guiding program may be configured to execute a method for assisting at least the preparation of a method of determining an interaction between a target compound and a test compound, comprising:
loading one or more of the following input parameters such as the properties and amounts of the metal nanoparticles (NPs), the target definition compound (TDC), the TDC-NP conjugate, the target compound, and the test compound; and
computing (and output or display) from those input parameters one or more of the remaining parameters needed to start the determining method.

In certain embodiments, the guiding program may be configured to execute a method for assisting at least the preparation of a method of identifying whether a compound is capable of modulating the interaction between a first polypeptide P1 and a second polypeptide P2, comprising:
loading one or more of the following input parameters such as the properties and amounts of the metal nanoparticles (NPs), P1, the P1-NP conjugate, P2, and the test compound; and
computing (and output or display) from those input parameters one or more of the remaining parameters needed to start the determining method.

The use of detergents for suppressing background signals as described above is particularly suitable for the specific methods of determining an interaction between a target compound and a test compound as described herein. However, the skilled person will understand that detergents may also be used for the suppression of background signals in other assay methods. Accordingly, further provided is a method for suppressing background signals in a method of determining an interaction between a test compound and a target compound, comprising providing said test compound in a solution comprising at least one detergent. Preferred detergents and detergent concentrations are described above. More particularly, provided herein is a method of determining an interaction between a target compound and a test compound, comprising:
(i) providing a suspension of a target definition compound (TDC) conjugated to metal nanoparticles (NPs) (TDC-NP conjugate), wherein said TDC can bind to said target compound;
(ii) contacting said suspension comprising said TDC-NP conjugate with said target compound and said test compound, wherein said test compound is provided in a solution comprising a detergent; thereby obtaining a liquid mixture comprising said TDC-NP conjugate, said test compound, said target compound, and said detergent; and
(iii) determining whether said test compound modulates binding of said target compound to said TDC, based on the presence or absence of a change in Localized Surface Plasmon Resonance (LSPR) properties of said TDC-NP conjugate when contacting said suspension comprising said TDC-NP conjugate with said target compound and said test compound.

The features of steps (a), (b), and (c) as described above apply, mutatis mutandis, for steps (i), (ii), and (iii), respectively. The presence of DMSO in the solutions comprising the test compound or target compound is optional. In particular embodiments, the concentration of the detergent in the liquid mixture obtained in step (b) is between 5 times and 10 times the CMC of the detergent.

The following examples are provided for the purpose of illustrating the present invention and by no means are meant and in no way should be interpreted to limit the scope of the present invention.

### Examples

### A) Determining of Interaction Between a Small Molecule and a Protein

Biotin is known to bind with high affinity to the protein Neutravidin. HABA ((2-(4-hydroxyazobenzene) benzoic acid)) binds to neutravidin in a similar way as biotin, but with lower affinity. The interaction between neutravidin and HABA was assessed using a method as described herein, using biotin as target definition compound (TDC).

### A1) Conjugation of Biotin to Gold Nanorods (GNRs)

Gold nanorods (GNRs) which are coated with mercaptoundecanoic acid (MUDA) were provided in suspension. The MUDA-coated GNRs provide an outer layer of carboxyl functional groups on their surface. For the conjugation of biotin to the GNRs, a biotin derivative (amino-PEG4-biotin) was used containing a polyethyleneglycol linker (4 monomers) having an amino functional group. More particularly, the carboxyl groups on the GNRs were activated using ethyl(dimethylaminopropyl) carbodiimide (EDC) and N-hydroxysuccinimide (NHS). Then, amino-PEG4-biotin was coupled via its amino functional group to the carboxyl groups provided on the GNRs, thereby providing a biotin-GNR conjugate. Potentially remaining unreacted carboxylic acid groups were blocked via reaction with 2-(2-aminoethoxy)ethanol (AEE). The biotin-GNR conjugate was purified from unreacted EDC, NHS, amino-PEG4-biotin and AEE by buffer exchange using a centrifugal ultrafiltration device.

### A2) Determining the Optimal Neutravidin Concentration to be Used

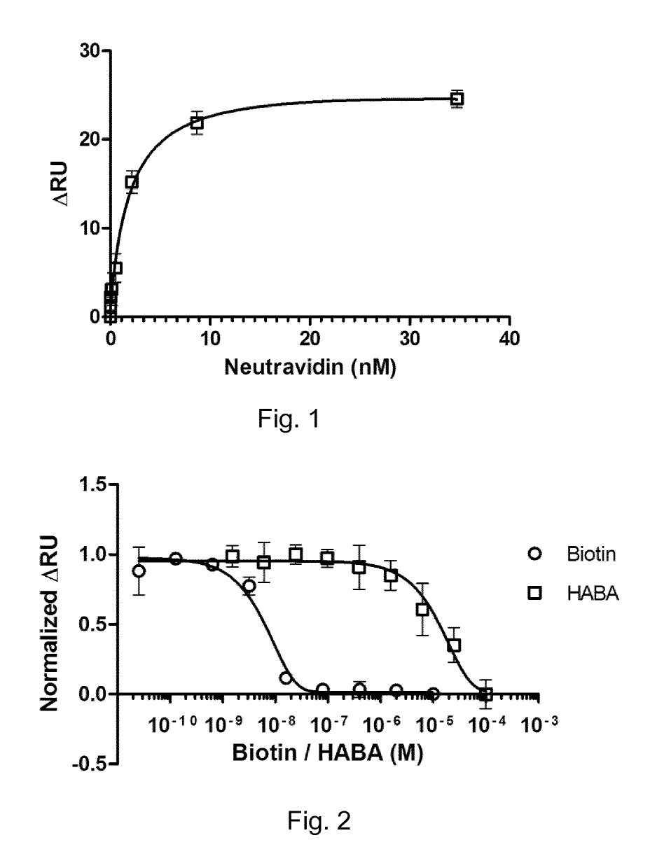

The determination of the inhibition of the interaction between HABA and Neutravidin according to the methods described herein involves contacting the biotin-GNR conjugate with Neutravidin. The optimal amount of Neutravidin to be contacted with the conjugate was determined via titration. More particularly, a fixed amount of biotin-GNR was incubated with various concentrations of Neutravidin, and the absorbance spectra after incubation was recorded. ΔRU was calculated and plotted as a function of the Neutravidin concentration (FIG. 1). Suitable Neutravidin concentrations are determined as those concentrations which are sufficiently high to provide a detectable signal (ΔRU), provided that the signal is not in the plateau of the dose-response curve. Optimal concentrations are those providing a signal in the linear response range.

### A3) Determining the Interaction Between HABA and Neutravidin

A fixed amount of Neutravidin was pre-incubated with different concentrations of HABA and biotin, followed by incubation with a fixed amount of biotin-GNR conjugate. HABA was provided as a solution in DMSO. The final DMSO concentration was 5 w %. The relative amounts of biotin-GNR and Neutravidin used are determined via titration as described above (A2). After incubation of Neutravidin with HABA/biotin and biotin-GNR, the absorbance spectra were recorded. ΔRU was calculated and plotted as a function of the HABA and biotin concentration (FIG. 2).

More particularly, ΔRU is calculated as RUₛₐₘₚₗₑ−RU_{blank}.; i.e. (OD(λ_{max;blank}+80)/OD(λ_{max, sample}))-(OD(λ_{max;blank}+80)/OD(λ_{max, blank})), wherein “OD(x)” refers to the optical density at wavelength x (in nm), and λₘₐₓ refers to the wavelength of maximal absorbance of the TDC-NP conjugate. RUₛₐₘₚₗₑ is the RU value for the sample (i.e. biotin-GNR sample with added neutravidin and HABA/biotin) and RU_{blank} is the RU value for the control sample (only biotin-GNR, in the same solvent comprising 5 w % DMSO).

The results show that a maximal ΔRU is obtained for low concentrations of HABA and biotin, indicating that Neutravidin binds to the biotin of the biotin-GNR conjugate. As the concentration of added biotin or HABA increases, ΔRU decreases, indicating that less Neutravidin binds to the biotin-GNR conjugate. This is because the HABA and biotin in solution competes with the biotin-GNR for binding with Neutravidin. For a significant reduction of ΔRU, a much higher concentration of HABA is needed compared to biotin, indicating that HABA has a lower affinity to Neutravidin than biotin.

### B) Inhibition of Protein-Protein Interactions

The p53 protein (also known as cellular tumor antigen p53) is known to bind with high affinity to the MDM2 protein (Mouse Double Minute 2 homolog). The compound nutlin-3 also binds to MDM2, thereby inhibiting further binding of MDM2 to p53. The inhibition of the p53-MDM2 interaction by nutlin-3 was assessed using a method as described herein.

### B1) Conjugation of p53 Peptide (Sequence: CGSGSGSGSGSRFMDYWEGL) to Gold Nanorods (GNRs)

Gold nanorods (GNRs) which are coated with mercaptoundecanoic acid (MUDA) were provided in suspension. The MUDA-coated GNRs provide an outer layer of carboxyl functional groups on their surface. p53 peptide was conjugated to the GNRs, using a N-(2-aminoethyl)maleimide linker. More particularly, the carboxyl groups were activated using EDC and NHS. Then, N-(2-aminoethyl)maleimide is coupled via its amino functional group to the carboxyl groups provided on the GNRs, thereby providing a layer of maleimide groups on the GNR surface. Potentially remaining unreacted carboxylic acid groups were blocked via reaction with AEE. The maleimide-functionalized GNRs were then purified from unreacted EDC, NHS, N-(2-aminoethyl)maleimide and AEE by buffer exchange using a centrifugal ultrafiltration device.

The p53 peptide was then coupled via its sulfhydryl group (provided by the N-terminal cysteine of p53) to the maleimide moieties on the GNR, thereby obtaining a p53-GNR conjugate. The pH of the GNR suspension during coupling was buffered at a pH of 7.5, which is above the pl of the p53 peptide (5.66). The p53-GNR conjugate was reacted with sulfhydryl functionalized methoxy polyethylene glycol (mPEG-SH), thereby blocking any remaining unreacted maleimide groups. The p53-GNR conjugate was purified from unreacted p53 peptide and mPEG-SH by buffer exchange using dialysis.

### B2) Determining the Optimal MDM2 Concentration to be Used

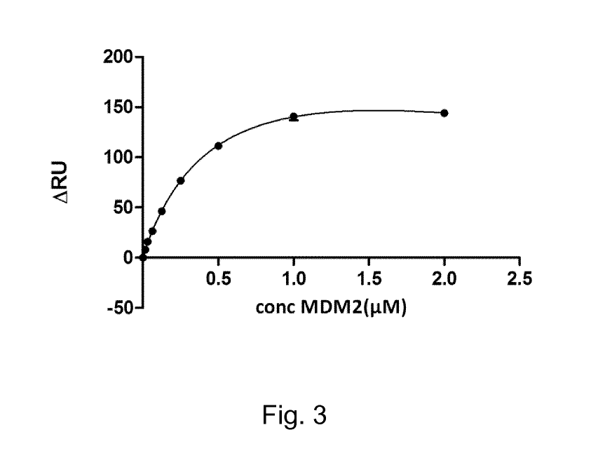

The determination of the inhibition of the interaction between MDM2 and p53 according to the methods described herein involves contacting the p53-GNR conjugate with MDM2. The optimal amount of MDM2 to be contacted with the conjugate was determined via a similar titration experiment as described above for Neutravidin (A2). ΔRU was calculated and plotted as a function of the MDM2 concentration (FIG. 3).

### B3) Determining the Interaction Between MDM2 and p53

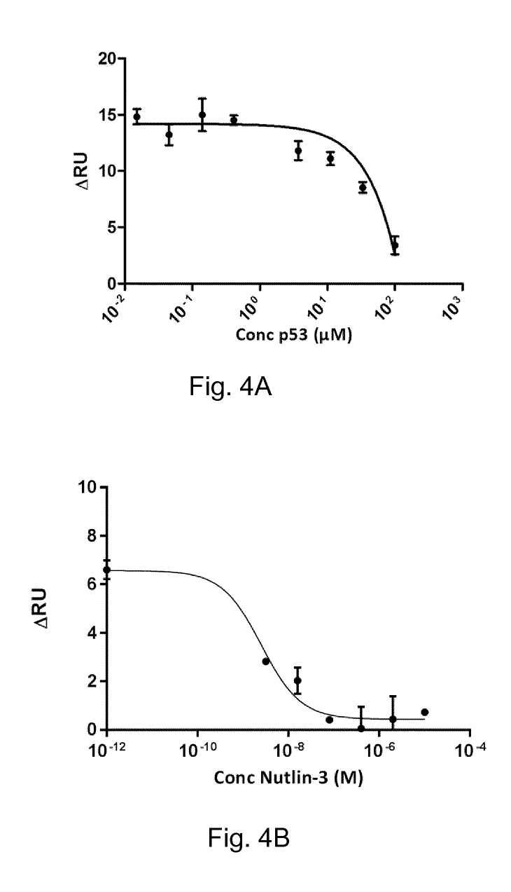

A fixed amount of MDM2 was pre-incubated with different concentrations of nutlin-3 or p53, followed by incubation with a fixed amount of p53-GNR conjugate. The relative amounts of p53-GNR and MDM2 used are determined via titration as described above (B2). After incubation of MDM2 with nutlin-3 (or p53) and p53-GNR, the absorbance spectra were recorded. ΔRU was calculated and plotted as a function of the added amount of nutlin-3 and p53 (FIGS. 4A and 4B).

Again, the results show a maximal ΔRU at low concentrations of added nutlin-3 and p53, indicating that MDM2 binds to the p53 of the p53-GNR conjugate. As the concentration of added nutlin-3 or p53 increases, ΔRU decreases, as the nutlin-3 and p53 in solution competes with the p53-GNR for binding to MDM2. For a significant reduction of ΔRU, a much higher concentration of p53 is needed compared to nutlin-3, indicating that nutlin-3 has a much higher affinity to MDM2 than p53.

The above examples show that the methods described herein may allow for the identification of compounds which modulate the interaction between two compounds, which may be small molecules or proteins.

### C) Suppression of Background Signal

Four small molecules (test compounds 1 to 4) were dissolved in DMSO, and subsequently added to a buffer comprising either no Triton X-100 or 0.1 volume % (v %), thereby obtaining liquid mixtures comprising 2 v % DMSO. For each of the test compounds, various solutions were prepared with increasing concentration of the test compound (0-100 μM). Subsequently, the solutions comprising the test compounds were incubated with a suspension of a TDC conjugated to GNRs (TDC-GNR; 1 v % final DMSO concentration), and absorbance spectra of the resulting suspensions was recorded. The experiments were performed in the absence of target compounds.

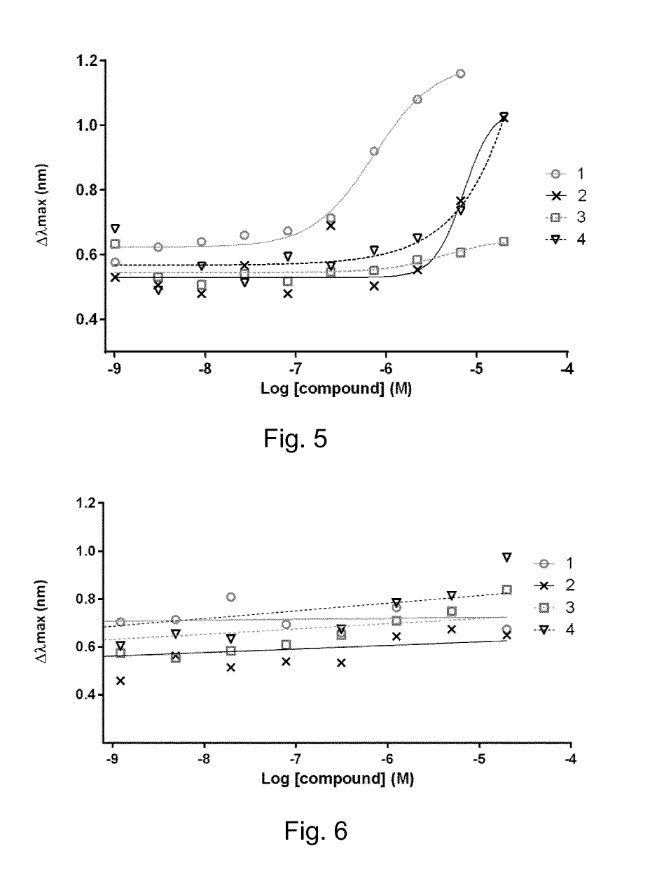

FIGS. 5 and 6 show the wavelength of maximal absorbance (Amax) for the suspensions without and with detergent (Triton X-100), respectively. The results shown that in the absence of detergent, Amax of the TDC-NP increases at higher compound concentrations. This is indicative of a (non-specific) interaction of the test compounds with the TDC at elevated test compound concentration and generates unwanted background signals. In contrast, no significant change of Amax is observed in the presence of 0.1 v % Triton X-100. Accordingly, these results show that background signals can be suppressed via the addition of a detergent such as Triton X-100.

1-15. (canceled)

16. A method of determining an interaction between a target compound and a test compound, comprising:
(a) providing a suspension of a target definition compound (TDC) conjugated to metal nanoparticles (NPs) (TDC-NP conjugate), wherein said TDC is defined as a compound which is known to bind to said target compound, wherein said metal nanoparticles are gold nanorods (GNR) and wherein said metal nanoparticles are at least partially coated with a linker molecule comprising a spacer group comprising a hydrocarbon chain with 6 to 18 carbon atoms, said spacer group terminated at one end with a functional group with a metal binding functionality, preferably sulfhydryl, and at another end with a functional group capable of forming a covalent bond to the TDC;
(b) contacting said suspension comprising said TDC-NP conjugate with said target compound and said test compound, wherein said test compound is provided in a solution comprising at least 50 w % DMSO; thereby obtaining a liquid mixture comprising a dimethylsulfoxide (DMSO) concentration below 50 w %; and
(c) determining whether said test compound modulates binding of said target compound to said TDC, based on the presence or absence of a change in refractive index surrounding the NPs due to the binding of said target compound to said TDC when contacting said suspension comprising said TDC-NP conjugate with said target compound and said test compound.

17. The method according to claim 16, wherein said target compound is a protein.

18. The method according to claim 16, wherein step (b) comprises:
(b1) incubating a solution of said target compound with said test compound; thereby obtaining a pre-incubated target compound solution comprising at least 0.5 w % DMSO; and
(b2) contacting said TDC-NP conjugate with said pre-incubated target compound solution.

19. The method according to claim 16, wherein step (c) comprises:
(c1) monitoring step (b) by illuminating said nanoparticles with at least one excitation light source and monitoring one or more optical properties of said nanoparticles; and
(c2) detecting a change in refractive index surrounding said nanoparticles wherein said change is a result of the presence of an interaction between said target compound and said TDC.

20. The method according to claim 19, wherein steps (c1) and (c2) are repeated at least once.

21. The method according to claim 16, wherein step (c) comprises correcting the change in LSPR properties of the TDC-NP conjugate for the presence of DMSO in said liquid mixture comprising a dimethylsulfoxide (DMSO) concentration below 50 w %.

22. The method according to claim 16, wherein said step (a) comprises:
(a1) providing a suspension of metal nanoparticles (NPs);
(a2) coupling said TDC to a linker molecule; and
(a3) conjugation of said TDC to said nanoparticles via said linker molecule, thereby obtaining a suspension comprising said TDC-NP conjugate.

23. The method according to claim 16, wherein said method further comprises determining the target compound concentration to be used in step (b) via a concentration titration of said TDC-NPs with said target compound, wherein a suitable amount of the target compound is an amount which results in a detectable change of the LSPR properties of the TDC-NPs but which does not saturate the available TDC binding sites.

24. The method according to claim 16, wherein said liquid mixture obtained in step (b) comprises between 0.5 w % and 10 w % DMSO.

25. The method according to claim 16, wherein said solution comprising said test compound further comprises a detergent.

26. The method according to claim 25, wherein the concentration of said detergent in said solution is above the critical micelle concentration.

27. A method of identifying a test compound capable of modulating the interaction between a first polypeptide P1 and a second polypeptide P2, comprising:
(A) providing a suspension of metal nanoparticles (NPs), wherein said metal NPs are provided with functional groups which carry negative charges and wherein said suspension of metal NPs is buffered at a pH between (pI-1) and pI, wherein pI is the isoelectric point of P1; conjugating P1 to said metal nanoparticles and providing a suspension of P1 conjugated to metal nanoparticles (NPs) (P1-NP conjugate);
(B) contacting said suspension comprising said P1-NP conjugate with P2 and a test compound; and
(C) determining whether said test compound modulates the interaction between P1 and P2, based on the presence or absence of a change in refractive index surrounding the NPs due to the binding of P1 to P2, when contacting said suspension comprising said P1-NP conjugate with P2 and said test compound.

28. The method according to claim 27, wherein said metal NPs in step (A) are provided with carboxyl groups.

29. The method of claim 28, wherein step (A) comprises:
(A1) providing a suspension of metal nanoparticles (NPs), wherein said suspension has a pH between (pI-1) and pI, wherein pI is the isoelectric point of P1;
(A2) coupling P1 to a linker molecule or coupling a linker molecule to said NPs; and
(A3) conjugation of P1 to said nanoparticles via said linker molecule, thereby obtaining a suspension comprising said P1-NP conjugate.

30. The method according to claim 29, wherein said linker molecule is coupled to P1 via a maleimide functional group.

31. A kit comprising:
a solution comprising a target compound and at least 50 w % DMSO; and
a suspension of a target definition compound (TDC) conjugated to metal nanoparticles (NPs) (TDC-NP conjugate), wherein the TDC is defined as a compound which is known to bind to the target compound and wherein said metal nanoparticles are at least partially coated with a linker molecule comprising a spacer group comprising a hydrocarbon chain with 6 to 18 carbon atoms, said spacer group terminated at one end with functional group with a metal binding functionality, preferably sulfhydryl, and at the other end with a functional group capable of forming a covalent bond to the TDC.
